(19) United States
(12) Patent Application Publication
(10) Pub. No.: US 2016/0074605 A1
(43) Pub. Date: Mar. 17, 2016

(54) GAS EXHAUST CONTROL METHOD AND RESPIRATORY ASSISTANCE APPARATUS APPLIED WITH THE SAME

(71) Applicant: DELTA ELECTRONICS, INC., Taoyuan City (TW)

(72) Inventors: Jung-Yu LIN, Taoyuan City (TW); Sheng-Wen PAI, Taoyuan City (TW); Chun-Chen CHEN, Taoyuan City (TW); Ren-De HUANG, Taoyuan City (TW); Shih-Kai CHIEN, Taoyuan City (TW); Chia-Ching LIN, Taoyuan City (TW)

(21) Appl. No.: 14/726,905

(22) Filed: Jun. 1, 2015

(30) Foreign Application Priority Data
Sep. 15, 2014 (TW) 103131710

(51) Int. Cl. A61M 16/00; A61M 16/06

(52) U.S. Cl. CPC A61M 16/0069; A61M 16/0003; A61M 16/06; A61M 2016/0027; A61M 2205/3334; A61M 2205/3546

(57) ABSTRACT

A gas exhaust control method applied to a respiratory assistance apparatus includes the following steps of: applying gas to a face mask of the respiratory assistance apparatus; detecting a pressure in the face mask; when the pressure in the face mask is lower than a preset pressure range, shrinking a perforation size of an exhaust structure of the face mask by a driving unit of the respiratory assistance apparatus; and when the pressure in the face mask is higher than the preset pressure range, expanding the perforation size of the exhaust structure of the face mask by the driving unit. A respiratory assistance apparatus applied with the gas exhaust control method is also disclosed.

### CROSS REFERENCE TO RELATED APPLICATIONS

This Non-provisional application claims priority under 35 U.S.C. §119(a) on Patent Application No(s). 103131710 filed in Taiwan, Republic of China on Sep. 15, 2014, the entire contents of which are hereby incorporated by reference.

### BACKGROUND OF THE INVENTION

1. Field of Invention

The present invention relates to a gas exhaust control method and a respiratory assistance apparatus applied with the gas exhaust control method.

2. Related Art

Continuous positive airway pressure (CPAP) machine is used for assisting users or patients breathing, especially for obstructive sleep apnea treatment. Continuous positive airway pressure machine is generally acknowledged to be the method with best healing efficacy.

Generally speaking, the existing continuous positive airway pressure machine outputs gas (e.g. oxygen) in the inhale mode. Gas is provided to users with stable pressure to increase the pressure within the user's respiratory tract and then provide into the user's lung. On the contrary, in the exhale mode, the continuous positive airway pressure machine stops outputting gas, and the gas generating from gas exchange process (e.g. carbon dioxide) in the mask is discharged out of the mask through the perforations on the mask to ensure that the carbon dioxide concentration in the mask is not excessively high. However, when the user is in the sleep mode, they may not breathe with stable pressure continuously. Hence, while using the continuous positive airway pressure machine in the sleep mode, users often breathe with unsmooth breathing condition. Personnel responsible for controlling the pressure of the continuous positive airway pressure machine are needed in the user's sleep mode, thus making the existing continuous positive airway pressure machine to cause great inconvenience to the user.

Moreover, the perforations on the mask of the existing continuous positive airway pressure machine may reduce pressure in the inhale mode and increase pressure in the exhale mode of the user, thus increasing the difficulty of pressure control.

So far, the air volume provided by the host engine (e.g. motor) can be controlled by detecting the respiratory condition of the user. However, while the respiratory condition is changing quickly, the host engine needs to quickly adjust its rotation speed which may speed up the consumption of host engine and make excessive noise, thus causing users trouble and inconvenience.

Accordingly, the present invention provides a gas exhaust control method and respiratory assistance apparatus applied the same to encounter the bottleneck of the conventional techniques.

### SUMMARY OF THE INVENTION

In view of the forgoing, an objective of the present invention is to provide a gas exhaust control method and a respiratory assistance apparatus that can control the exhaust structure of the face mask for adjusting the pressure inside the face mask under a constant air supply, thereby effectively improving the utility of the respiratory assistance apparatus.

To achieve the above objective, the present invention discloses a gas exhaust control method applied to a respiratory assistance apparatus. The gas exhaust control method includes the following steps of: applying gas to a face mask of the respiratory assistance apparatus; detecting the respiratory status of a user wearing the face mask; when the respiratory status of the user is inspiration, shrinking the perforation size of an exhaust structure of the face mask by a driving unit of the respiratory assistance apparatus; and when the respiratory status of the user is expiration, expanding the perforation size of the exhaust structure of the face mask by the driving unit.

In one embodiment, the respiratory status further includes the breath amplitude of the user.

In one embodiment, the perforation size of the exhaust structure is fixed after being shrunk or expanded.

In one embodiment, when the respiratory status of the user is inspiration, the perforation size of the exhaust structure is shrunk to completely close.

In one embodiment, when the respiratory status of the user is expiration, the perforation size of the exhaust structure is expanded to completely open.

In one embodiment, the driving unit is a solenoid valve or a motor.

In one embodiment, the perforation size of the exhaust structure is shrunk or expanded by rotating or pulling.

In one embodiment, the gas exhaust control method further includes the steps of: when the respiratory status of the user is inspiration, introducing an airflow through the exhaust structure by a blowing unit of the respiratory assistance apparatus; and when the respiratory status of the user is expiration, outputting an airflow through the exhaust structure by the blowing unit.

In one embodiment, the blowing unit is a blower.

To achieve the above objective, the present invention also discloses a gas exhaust control method applied to a respiratory assistance apparatus. The gas exhaust control method includes the following steps of: applying gas to a face mask of the respiratory assistance apparatus; detecting a pressure in the face mask; when the pressure in the face mask is lower than a preset pressure range, shrinking a perforation size of an exhaust structure of the face mask by a driving unit of the respiratory assistance apparatus; and when the pressure in the face mask is higher than the preset pressure range, expanding the perforation size of the exhaust structure of the face mask by the driving unit.

In one embodiment, when the pressure in the face mask is lower than the preset pressure range, the perforation size of the exhaust structure is shrunk to completely close.

In one embodiment, when the pressure in the face mask is higher than the preset pressure range, the perforation size of the exhaust structure is expanded to completely open.

In one embodiment, the perforation size of the exhaust structure is shrunk or expanded by rotating or pulling.

To achieve the above objective, the present invention further discloses a gas exhaust control method applied to a respiratory assistance apparatus. The gas exhaust control method includes the following steps of: applying gas to a face mask of the respiratory assistance apparatus; detecting a respiratory status of a user wearing the face mask; when the respiratory status of the user is inspiration, decreasing an exhaust amount of the face mask by a driving unit of the respiratory assistance apparatus; and when the respiratory status of the user is expiration, increasing the exhaust amount of the face mask by the driving unit.

To achieve the above objective, the present further discloses a respiratory assistance apparatus including a face mask, a channel, a gas supply module and an exhaust control module. The face mask has an exhaust structure. The channel is connected to the face mask, and the gas supply module is connected to the channel. The gas supply module includes a first processing unit, a gas supply unit coupled to the first processing unit, and a fluid measuring unit coupled to the as supply unit. The exhaust control module includes a second processing unit, a pressure sensing unit and a driving unit. The second processing unit is coupled to the first processing unit, the pressure sensing unit is coupled to the second processing unit, and the driving unit is coupled to the second processing unit. The driving unit shrinks or expands a perforation size of the exhaust structure.

In one embodiment, the gas supply module further includes a pressure sensor and an air quantity sensor, and the pressure sensor and the air quantity sensor are connected to the first processing unit and the fluid measuring unit.

In one embodiment, the first processing unit and the second processing unit are coupled by wired connection or wireless connection.

In one embodiment, the fluid measuring unit is a Venturi tube, a Pitot tube or a honeycomb structure.

In one embodiment, the driving unit is a solenoid valve, a motor or a blower.

In one embodiment, the exhaust structure has at least two exhaust units.

In one embodiment, the driving unit is coupled to the exhaust units.

In one embodiment, the driving unit rotates or pulls the exhaust units.

In one embodiment, the face mask has a blowing unit disposed on the exhaust units.

In one embodiment, the blowing unit is a blower.

To achieve the above objective, the present invention also discloses a respiratory assistance apparatus including a face mask, a channel, a gas supply module and an exhaust control module. The face mask has an exhaust structure. The channel is connected to the face mask, and the gas supply module is connected to the channel. The exhaust control module is coupled to the gas supply module. The exhaust control module includes a driving unit for shrinking or expanding a perforation size of the exhaust structure.

In one embodiment, the exhaust structure has at least two exhaust units.

In one embodiment, the driving unit is coupled to the exhaust units.

In one embodiment, the driving unit rotates or pulls the exhaust units.

As mentioned above, the gas exhaust control method and respiratory assistance apparatus of the invention can control the exhaust structure of the face mask for adjusting the pressure inside the face mask under a constant air supply, thereby effectively improving the utility of the respiratory assistance apparatus.

Regarding to the conventional respiratory assistance apparatus (e.g. CPAP) machine, it is necessary to adjust the motor to change the supplied air quantity. When the respiratory status of the user is rapidly changed, the motor must provide a quick adjustment in rotation speed. This quick adjustment can speed the wearing of the motor and generate a loud noise, which causes the uncomfortable and inconvenience of the user. The gas exhaust control method and respiratory assistance apparatus of the invention utilize the exhaust control module to effectively detect the respiratory status of the user and the pressure inside the face mask. Accordingly, the exhaust structure of the face mask can be properly adjusted in cooperate with the breath amplitude of the user. Thus, the noise of the conventional respiratory assistance apparatus can be minimized, and it is possible to adjust the apparatus based on the respiratory status of individual user, thereby improving the application of the entire apparatus.

### BRIEF DESCRIPTION OF THE DRAWINGS

The present invention will become more fully understood from the subsequent detailed description and accompanying drawings, which are given by way of illustration only, and thus are not limitative of the present invention, and wherein:

FIG. 1 is a flow chart of a gas exhaust control method according to a preferred embodiment of the invention;

FIG. 2A is a block diagram showing components of a respiratory assistance apparatus according to a preferred embodiment of the invention;

FIG. 2B is a perspective view showing a part of the respiratory assistance apparatus of FIG. 2A;

FIG. 2C is an exploded view of the part of the respiratory assistance apparatus of FIG. 2B;

FIG. 2D is a perspective view showing the exhaust unit of the exhaust structure shown in FIG. 2C;

FIG. 3A is a perspective view showing the exhaust unit of the exhaust structure of a respiratory assistance apparatus according to another embodiment of the invention;

FIG. 3B is a perspective view showing the exhaust unit of the exhaust structure of a respiratory assistance apparatus according to another embodiment of the invention;

FIG. 4 is an exploded view of another aspect of the face mask of the respiratory assistance apparatus;

FIG. 5 is a flow chart of a gas exhaust control method according to another embodiment of the invention; and

FIG. 6 is a flow chart of a gas exhaust control method according to another embodiment of the invention.

### DETAILED DESCRIPTION OF THE INVENTION

This invention discloses a gas exhaust control method and a respiratory assistance apparatus applied with the control method for improving the application of the respiratory assistance apparatus (e.g. a continuous positive airway pressure (CPAP) machine). The present invention will be apparent from the following detailed description, which proceeds with reference to the accompanying drawings, wherein the same references relate to the same elements.

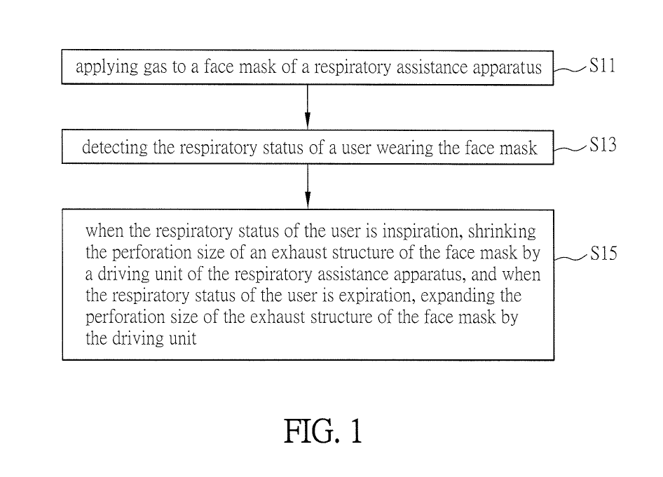

FIG. 1 is a flow chart of a gas exhaust control method according to a preferred embodiment of the invention. With reference to FIG. 1, the gas exhaust control method includes the following steps of: applying gas to a face mask of a respiratory assistance apparatus (S11); detecting the respiratory status of a user wearing the face mask (S13); and when the respiratory status of the user is inspiration, shrinking the perforation size of an exhaust structure of the face mask by a driving unit of the respiratory assistance apparatus, and when the respiratory status of the user is expiration, expanding the perforation size of the exhaust structure of the face mask by the driving unit (S15).

In order to make the details of the steps of the control method more clear, the application and structure of the respiratory assistance apparatus of the invention will be discussed in advance. Based on the respiratory assistance apparatus, the gas exhaust control method applied to the respiratory assistance apparatus will be described later. To be noted, the following embodiments are for illustrations only and are not to limit the scope of the invention.

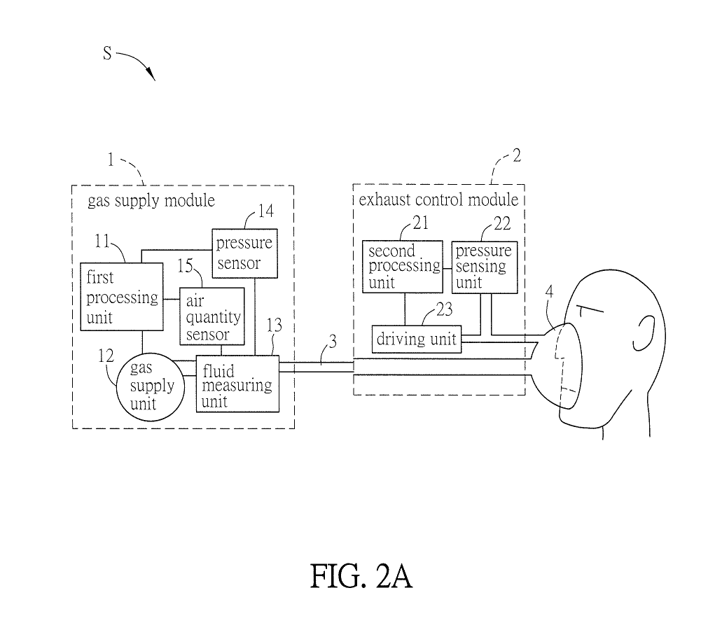

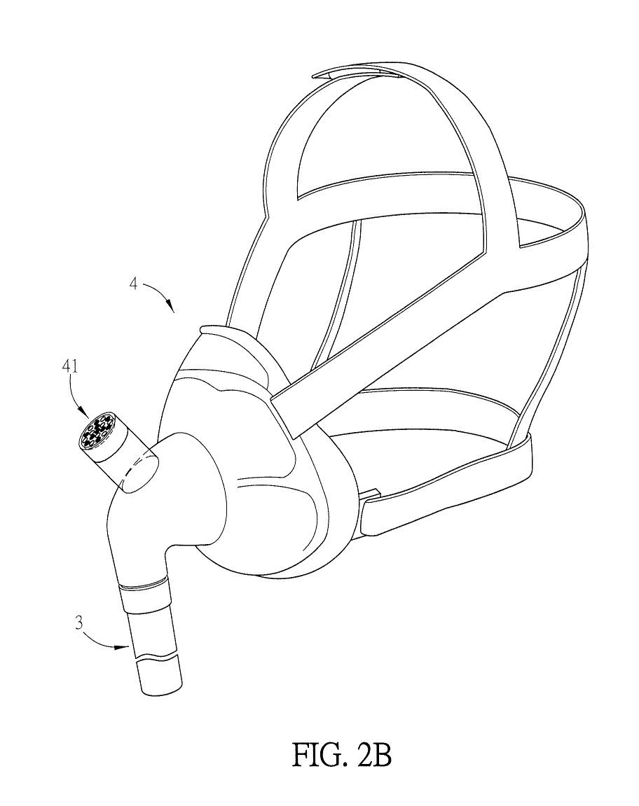

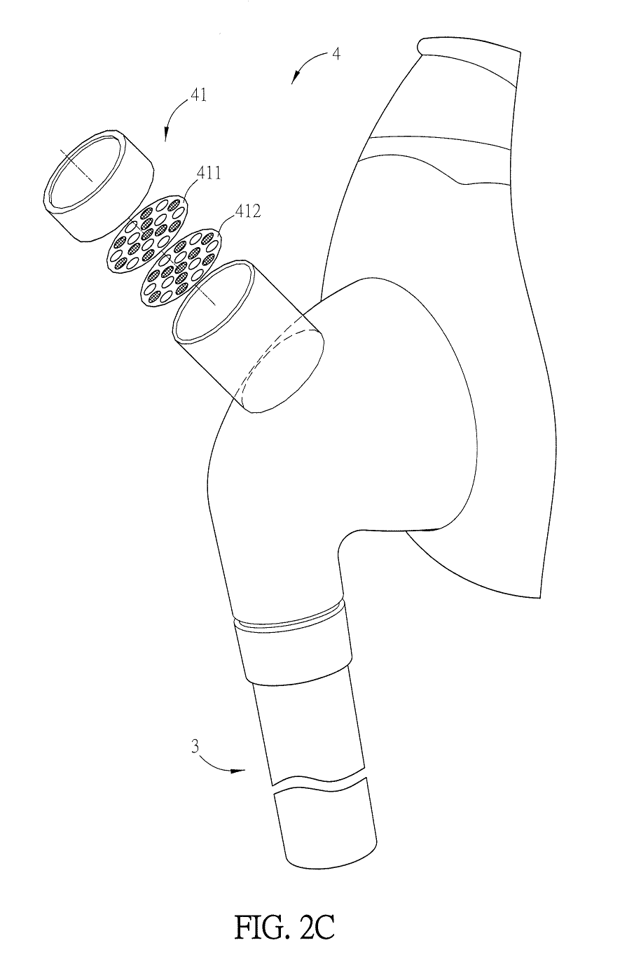

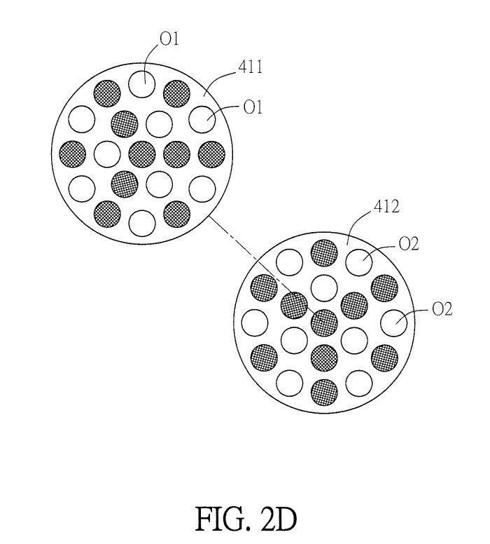

FIG. 2A is a block diagram showing components of a respiratory assistance apparatus according to a preferred embodiment of the invention, and FIG. 2B is a perspective view showing a part of the respiratory assistance apparatus of FIG. 2A. FIG. 2A helps to illustrate the complete circuits and connections of all components in the respiratory assistance apparatus S, and FIG. 2B shows the real appearance of the face mask 4. With reference to FIGS. 1, 2A and 2B, in the step S11, the respiratory assistance apparatus S of the embodiment includes a gas supply module 1, an exhaust control module 2, a channel 3 and a face mask 4. Herein, the respiratory assistance apparatus S is a CPAP machine for example. Two ends of the channel 3 connect to the gas supply module 1 and the face mask 4, respectively. Accordingly, the gas (e.g. oxygen) provided from the gas supply module 1 can be supplied to the face mask 4 through the channel 3. The exhaust control module 2 is coupled to the gas supply module 1 and the face mask 4 for controlling the air quantity inside the face mask 4.

In more detailed, the gas supply module 1 has a first processing unit 11, a gas supply unit 12, a fluid measuring unit 13, a pressure sensor 14 and an air quantity sensor 15. The first processing unit 11 is a microcontroller unit (MCU), and the gas supply unit 12 is a blower. The first processing unit is coupled to the gas supply unit 12, the pressure sensor 14 and the air quantity sensor 15. The first processing unit 11 can control the rotation speed of the gas supply unit 12. In this embodiment the gas supply unit 12 is operated at a constant rotation speed for providing a fixed air quantity. The gas provided by the gas supply unit 12 flows through the fluid measuring unit 13, and the first processing unit 11 can measure the air quantity flowing through the fluid measuring unit 13 by the pressure sensor 14 and the air quantity sensor 15. Accordingly, the first processing unit 11 can control the operation of the gas supply unit 12 to increase the air quantity until the desired air pressure. The stable and desired air quantity can be provided based on the control of the first processing unit 11.

To be noted, the fluid measuring unit 13 can be a Venturi tube, a Pitot tube or a honeycomb structure, and this invention is not limited thereto.

Then, the gas provided by the gas supply unit 12 flows to the face mask 4 through the channel 3. In the step S13, the gas exhaust control module 2 also detects the respiratory status of a user wearing the face mask 4. The respiratory status includes inspiration/expiration status and the breath amplitude. In more detailed, the gas exhaust control module 2 includes a second processing unit 21, a pressure sensing unit 22 and a driving unit 23. The second processing unit 21 is also a microcontroller unit for determining whether the user is in an inspiration status or an expiration status. The pressure sensing unit 22 is configured to detect the breath amplitude of the user and transmits this information to the second processing unit 21. The second processing unit 21 controls the driving unit 23 to operate according to the amplitude detected by the pressure sensing unit 22.

In more detailed, the driving unit 23 is controlled by the second processing unit 21 so as to adjust the air input or air output of the face mask 4. The structure of the face mask 4 can be referred to FIGS. 2C and 2D, wherein FIG. 2C is an exploded view of the part of the respiratory assistance apparatus of FIG. 2B, and FIG. 2D is a perspective view showing the exhaust unit of the exhaust structure shown in FIG. 2C. Referring to FIGS. 1 to 2D, the face mask 4 has an exhaust structure 41, which has at least two exhaust units 411 and 412. The exhaust units 411 and 412 are relatively movable and engaged to the face mask 4. For example, the exhaust units 411 and 412 can be relatively rotated or relatively moved. The two exhaust units 411 and 412 have the same or different through holes. In this embodiment, the exhaust unit 411 has a plurality of through holes O1, and the exhaust unit 412 has a plurality of through holes O2. Wherein, the configuration relationship of the through holes O1 and the through holes O2 are the same. The driving unit 23 of this embodiment is coupled to the two exhaust units 411 and 412. The relative positions of the through holes O1 and O2 can be adjusted by rotating or pulling the exhaust units 411 and 412, thereby forming different perforation sizes of the exhaust structure 41.

To be noted, the perforation size of the exhaust structure 41 is the total volume of the through holes that allows the airflow passing through the face mask 4 as the through holes O1 and O2 of the exhaust units 411 and 412 are located at relative positions. In other words, the perforation size formed by the misaligned through holes O1 and O2 can be shrunk or expanded by adjusting the relative positions of the through holes O1 of the exhaust unit 411 and the through holes O2 of the exhaust unit 412.

Furthermore, in the step S15, when the second processing unit 21 determines that the respiratory status of the user is inspiration, the second processing unit 21 commands the driving unit 23 to shrink the perforation size of the exhaust structure 41. On the contrary, when the second processing unit 21 determines that the respiratory status of the user is expiration, the second processing unit 21 commands the driving unit 23 to expand the perforation size of the exhaust structure 41. In this embodiment, the driving unit 23 can adjust the positions of the exhaust units 411 and 412 by, for example but not limited to, rotating or pulling. In practice, the driving unit 23 is a solenoid valve or a motor, and this invention is not limited thereto.

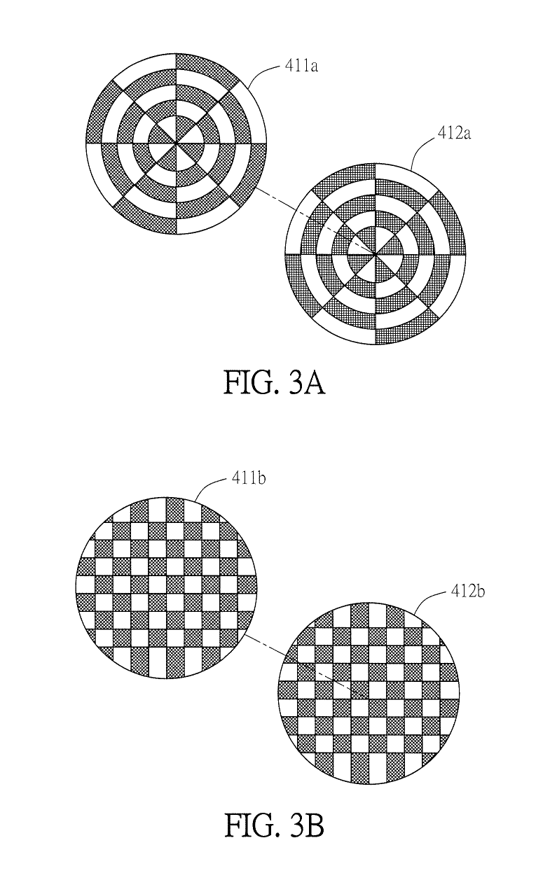

The number and type of the above-mentioned exhaust units 411 and 412 are not limited. In other embodiment, the exhaust structure may include two or more exhaust units, and the shape of the exhaust unit can be those shown in FIG. 3A (exhaust units 411a and 412a) or FIG. 3B (exhaust units 411b and 412b), or their modifications. As the driving unit controls to misalign two or more exhaust units, the total volume of air output can be properly adjust so as to achieve the goal of air quantity control.

In this embodiment, the perforation size of the exhaust structure is fixed after being shrunk or expanded in step S15. In other words, after the pressure sensing unit 22 detects the breath amplitude, the second processing unit 21 commands the driving unit 23 to shrink or expand the perforation size of the exhaust structure 41 and then maintain the perforation size of the exhaust structure 41 at the adjusted perforation size. The adjusted perforation size of the exhaust structure 41 is determined by the second processing unit 21 as a suitable size for the respiratory status of the user. Accordingly, the driving unit 23 is in a steady status, and it is unnecessary to change the positions of the exhaust units 411 and 412 all the time. This configuration can reduce the wearing of the driving unit 23 and the exhaust units 411 and 412, thereby effectively minimizing the noise as the motor or the exhaust units 411 and 412 operate.

In order to allow the respiratory assistance apparatus S to provide the best and most comfortable exhaust mechanism to the user, in other embodiments, the second processing unit 21 can command the driving unit 23 to shrink the perforation size of the exhaust structure 41 to complete close, when the respiratory status of the user is inspiration. On the contrary, the second processing unit 21 can command the driving unit 23 to expand the perforation size of the exhaust structure 41 to complete open, when the respiratory status of the user is expiration. That is, the perforation size of the exhaust structure 41 is not limited in the invention, and the user can have the most comfortable experience depending on the actual situation.

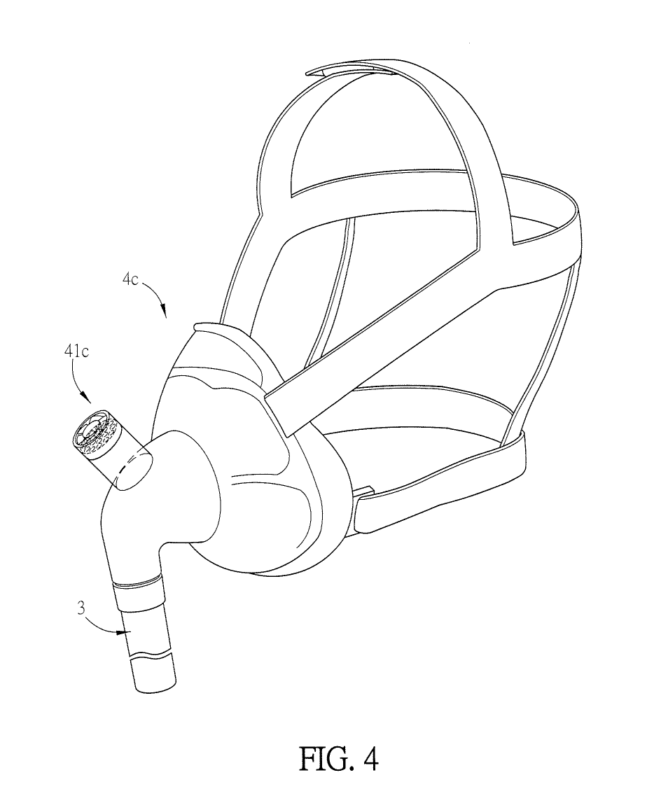

In other embodiments, the face mask may also include a blowing unit such as a blower. FIG. 4 is an exploded view of another aspect of the face mask of the respiratory assistance apparatus. In more detailed, with reference to FIGS. 2A and 4, the blowing unit is disposed at the position of the exhaust unit 41c. When the respiratory status of the user is inspiration, the second processing unit 21 commands the blowing unit to introduce airflow from outside through the exhaust structure 41c of the mask 4c, thereby avoiding the pressure drop during the inspiration of the user. On the contrary, when the respiratory status of the user is expiration, the second processing unit 21 commands the blowing unit to output the airflow through the exhaust structure 41c, thereby avoiding the pressure increase during the expiration of the user.

In practice, the gas exhaust control method of the invention can be applied to a bi-level positive airway pressure (BiPAP) to achieve the pressure control in dual stages of inspiration and expiration.

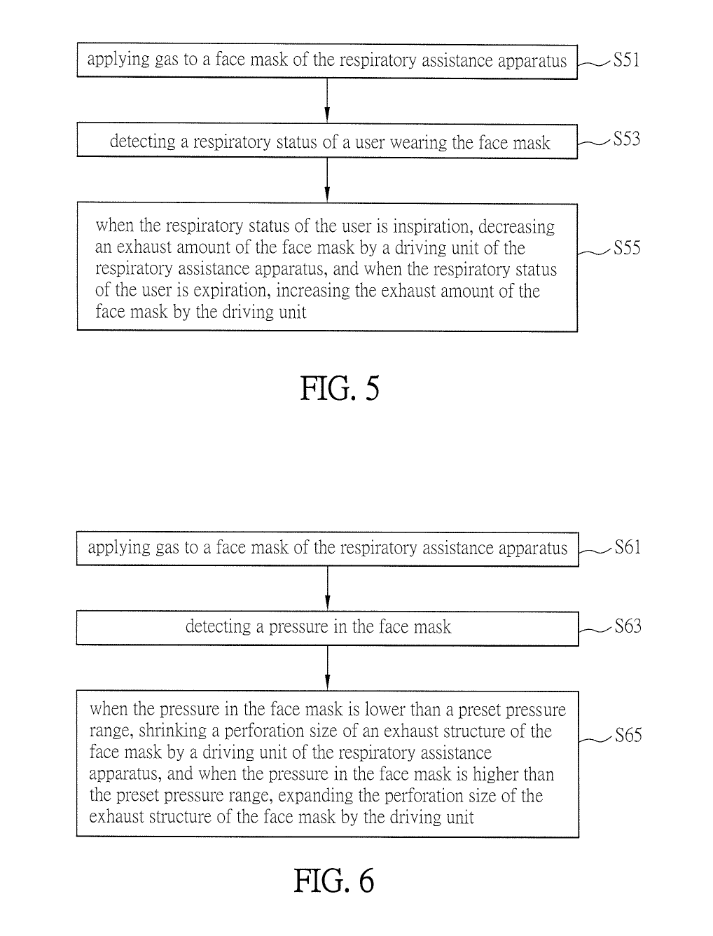

The present invention also discloses another gas exhaust control method applied to the above-mentioned respiratory assistance apparatus. With reference to FIG. 5, the gas exhaust control method includes the following steps of: applying gas to a face mask of the respiratory assistance apparatus (S51); detecting a respiratory status of a user wearing the face mask (S53); and when the respiratory status of the user is inspiration, decreasing an exhaust amount of the face mask by a driving unit of the respiratory assistance apparatus, and when the respiratory status of the user is expiration, increasing the exhaust amount of the face mask by the driving unit (S55). The details of the gas exhaust control method of FIG. 5 is mostly the same as that of FIG. 1, so the detailed description thereof will be omitted.

To be noted, the gas exhaust control method of this embodiment does not limit the method for controlling the exhaust amount of the mask by the driving unit, and it is possible to design according to the actual requirements.

The present invention also discloses another gas exhaust control method applied to the above-mentioned respiratory assistance apparatus. With reference to FIG. 6, the gas exhaust control method includes the following steps of: applying gas to a face mask of the respiratory assistance apparatus (S61); detecting a pressure in the face mask (S63); and when the pressure in the face mask is lower than a preset pressure range, shrinking a perforation size of an exhaust structure of the face mask by a driving unit of the respiratory assistance apparatus, and when the pressure in the face mask is higher than the preset pressure range, expanding the perforation size of the exhaust structure of the face mask by the driving unit (S65).

Referring to FIGS. 2A and 6, in the step S63, the pressure sensing unit 22 detects the pressure inside the face mask 4 and transmits the information to the second processing unit 21. The second processing unit 21 controls the operation of the driving unit 23 according to the pressure inside the face mask 4 detected by the pressure sensing unit 22 so as to execute the step S65.

In the step S65, when the pressure in the face mask 4 is lower than a preset pressure range, by the driving unit 23 of the respiratory assistance apparatus S shrinks the perforation size of the exhaust structure of the face mask 4; otherwise, when the pressure in the face mask 4 is higher than the preset pressure range, the driving unit 23 expands the perforation size of the exhaust structure. The preset pressure range is not limited in this embodiment, and it can be determined according to the requirement of the user (e.g. the pressure for therapy or the body condition of the user).

In summary, the gas exhaust control method and respiratory assistance apparatus of the invention can control the exhaust structure of the face mask for adjusting the pressure inside the face mask under a constant air supply, thereby effectively improving the utility of the respiratory assistance apparatus.

Regarding to the conventional respiratory assistance apparatus (e.g. CPAP machine), it is necessary to adjust the motor to change the supplied air quantity. When the respiratory status of the user is rapidly changed, the motor must provide a quick adjustment in rotation speed. This quick adjustment can speed the wearing of the motor and generate a loud noise, which causes the uncomfortable and inconvenience of the user. The gas exhaust control method and respiratory assistance apparatus of the invention utilize the exhaust control module to effectively detect the respiratory status of the user and the pressure inside the face mask. Accordingly, the exhaust structure of the face mask can be properly adjusted in cooperate with the breath amplitude of the user. Thus, the noise of the conventional respiratory assistance apparatus can be minimized, and it is possible to adjust the apparatus based on the respiratory status of individual user, thereby improving the application of the entire apparatus.

Although the present invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the present invention.

What is claimed is:

1. A gas exhaust control method, which is applied to a respiratory assistance apparatus, comprising steps of:
applying gas to a face mask of the respiratory assistance apparatus;
detecting a pressure in the face mask;
when the pressure in the face mask is lower than a preset pressure range, shrinking a perforation size of an exhaust structure of the face mask by a driving unit of the respiratory assistance apparatus; and
when the pressure in the face mask is higher than the preset pressure range, expanding the perforation size of the exhaust structure of the face mask by the driving unit.

2. The control method of claim 1, wherein when the pressure in the face mask is lower than the preset pressure range, the perforation size of the exhaust structure is shrunk to completely close.

3. The control method of claim 1, wherein when the pressure in the face mask is higher than the preset pressure range, the perforation size of the exhaust structure is expanded to completely open.

4. The control method of claim 1, wherein the perforation size of the exhaust structure is shrunk or expanded by rotating or pulling.

5. A gas exhaust control method applied to a respiratory assistance apparatus, comprising steps of:
applying gas to a face mask of the respiratory assistance apparatus;
detecting a respiratory status of a user wearing the face mask;
when the respiratory status of the user is inspiration, decreasing an exhaust amount of the face mask by a driving unit of the respiratory assistance apparatus; and
when the respiratory status of the user is expiration, increasing the exhaust amount of the face mask by the driving unit.

6. A respiratory assistance apparatus, comprising:
a face mask having an exhaust structure;
a channel connected to the face mask;
a gas supply module connected to the channel; and
an exhaust control module coupled to the gas supply module, wherein the exhaust control module comprises a driving unit for shrinking or expanding a perforation size of the exhaust structure.

7. The respiratory assistance apparatus of claim 6, wherein the exhaust structure has at least two exhaust units.

8. The respiratory assistance apparatus of claim 7, wherein the driving unit is coupled to the exhaust units.

9. The respiratory assistance apparatus of claim 7, wherein the driving unit rotates or pulls the exhaust units.

10. The respiratory assistance apparatus of claim 6, wherein the gas supply module comprises a first processing unit, a gas supply unit and a fluid measuring unit, the gas supply unit coupled to the first processing unit, the fluid measuring unit coupled to the gas supply unit, the exhaust control module further comprises a second processing unit and a pressure sensing unit, the second processing unit coupled to the first processing unit, the pressure sensing unit coupled to the second processing unit, and the driving unit coupled to the second processing unit.

11. The respiratory assistance apparatus of claim 10, wherein the gas supply module further comprises a pressure sensor and an air quantity sensor, and the pressure sensor and the air quantity sensor are connected to the first processing unit and the fluid measuring unit.

12. The respiratory assistance apparatus of claim 10, wherein the first processing unit and the second processing unit are coupled by wired connection or wireless connection.

13. The respiratory assistance apparatus of claim 10, wherein the fluid measuring unit is a Venturi tube, a Pitot tube or a honeycomb structure.

14. The respiratory assistance apparatus of claim 10, wherein the driving unit is a solenoid valve, a motor or a blower.

15. The respiratory assistance apparatus of claim 10, wherein the exhaust structure has at least two exhaust units.

16. The respiratory assistance apparatus of claim 15, wherein the driving unit is coupled to the exhaust units.

17. The respiratory assistance apparatus of claim 15, wherein the driving unit rotates or pulls the exhaust units.

18. The respiratory assistance apparatus of claim 15, wherein the face mask has a blowing unit disposed on the exhaust units.

19. The respiratory assistance apparatus of claim 18, wherein the blowing unit is a blower.
